(19) United States
(12) Patent Application Publication
(10) Pub. No.: US 2024/0350195 A1
(43) Pub. Date: Oct. 24, 2024

(54) SURGICAL PROBE DEVICE, AND SYSTEMS AND METHODS FOR COOLING THE SAME

(71) Applicant: Monteris Medical Corporation, Minnetonka, MN (US)

(72) Inventors: Mark Andrew Grant, Winnipeg (CA); Walter John Dobrovolny, Saint Paul, MN (US); Samuel Skoglund, East Bethel, MN (US); Jeffrey Allen, Londonberry, NH (US); Kellie Crawford, Plymouth, MN (US); Richard Tyc, Winnipeg (CA)

(21) Appl. No.: 18/642,684

(22) Filed: Apr. 22, 2024

Related U.S. Application Data
(60) Provisional application No. 63/497,407, filed on Apr. 20, 2023.

(51) Int. Cl. A61B 18/22; A61B 18/00; A61B 18/02; A61B 90/00

(52) U.S. Cl. CPC A61B 18/22; A61B 18/0218; A61B 90/03; A61B 2018/00577; A61B 2018/00589; A61B 2018/00714; A61B 2018/00791; A61B 2018/2261; A61B 2018/2266; A61B 2090/036

(57) ABSTRACT

Disclosed scenarios include a temperature modulated reduced-diameter probe assembly that includes a probe portion for effecting at least one of thermal therapy and cryotherapy to the tissue and having a diameter of about 1.65 mm or less. The probe portion includes a laser fiber configured for transmitting optical energy along its length, and a diffuser element at a distal tip of the laser fiber and configured for diffusing optical energy received from the distal tip of the laser fiber.

### RELATED APPLICATIONS AND CLAIMS OF PRIORITY

The current application claims priority to U.S. Provisional Application No. 63/497,407 filed Feb. 20, 2024, the disclosure of which is incorporated herein in its entirety.

### FIELD OF THE DISCLOSURE

The present disclosure generally relates small diameter surgical probe devices, and systems and methods for thermal treatment of diseased tissues. More specifically, the present disclosure relates to devices and ways to cool tissue in the vicinity of the diseased tissue being treated by said thermal treatment.

### BACKGROUND OF THE DISCLOSURE

Energy-based tissue treatment is well known in the art. Various types of energy (e.g., electrical, ultrasonic, microwave, cryogenic, thermal, laser, etc.) are applied to tissue to achieve a desired result (e.g., coagulate and/or cut, tissue). Generally, a target volume of tissue is sufficiently heated to achieve a therapeutic effect, such as thermal coagulation. Tissue thermal coagulation depends on a number of factors, and temperatures in the range of 55-60° C. are generally considered sufficient to provide enough energy to cause such coagulation. Cell death results immediately from heating to these temperatures and also occurs at lower temperatures (45-55° C.) for extended duration, and a region of irreversible thermal damage can be observed with imaging following the treatment. In addition, heating can be produced from minimallyinvasive applicators, eliminating the need for open surgery, and potentially reducing recovery time and morbidity for patients.

Interstitial thermal therapy is currently practiced by inserting heating applicators directly into a target site within an organ. Several energy sources have been integrated into interstitial heating applicators, including lasers, ultrasound, microwave, and radiofrequency energy. Preferably, interstitial thermal therapy delivers sufficient thermal energy to coagulate an entire target volume, while avoiding undesirable thermal damage to adjacent normal tissues. This approach has been used for treating brain lesions by which brain lesions are heated from within (e.g., using, laser interstitial thermal therapy (LITT) procedures).

In performing thermal treatments as described above, it is usually preferable to avoid damage to normal (non-diseased) tissue due to heating of the normal tissue in the vicinity of the diseased tissue. This is of special concern for normal tissues proximal to or in the vicinity of the treatment area (sometimes called a target volume or treatment zone) where heat is applied to the diseased tissue. In the example of thermal treatment of the brain, ultrasound, electromagnetic, RF, microwave, and other sources of heat can lead to heating of normal (non-diseased) tissue surrounding the target tissue or adjacent thereto. Excessive heating of this normal tissue could cause unwanted damage to the normal tissue.

Various attempts to prevent over-heating of tissue outside the targeted diseased tissue have included attempts to confine the volume within which the thermal therapy is applied so that thermal effects are reduced beyond the localized volume being treated. This solution can result in slower or less effective treatment, as treatment of an extended region in space would require application of many such localized treatments to a small treatment zone to avoid spreading the thermal energy to normal tissues outside the treatment zone. Other attempts to account for the heating of normal tissues outside a target volume use the cooling effects of time so that short duration pulses of heat are applied to a target area in order to avoid over-heating of various locations and to enable conduction or other heat transfer mechanisms to keep the temperature of normal tissues in check. Yet other attempts to counter the effect above include using the heat transfer capabilities of perfused tissue having blood flowing therethrough to carry away certain doses of heat applied to the tissue.

Therefore, it remains needed or useful to develop techniques and treatment systems to heat the diseased tissues in a targeted treatment volume sufficiently while at the same time avoiding over-heating of the proximal tissues and organs. In particular, the present embodiments and concepts will illustrate to one skilled in the art methods and apparatus for achieving an effective, robust, efficient thermal treatment of diseased tissue simultaneously with no, little, or reduced risk of damage to nearby normal tissues and organs under given conditions.

The current disclosure describes devices and methods directed towards solving some of the issues discussed above.

### SUMMARY OF THE DISCLOSURE

Disclosed scenarios include a temperature modulated reduced-diameter probe assembly that includes a probe portion for effecting at least one of thermal therapy and cryotherapy to the tissue and having a diameter of about 1.65 mm or less. The probe portion may include a laser fiber configured for transmitting optical energy along its length, and a diffuser element at a distal tip of the laser fiber and configured for diffusing optical energy received from the distal tip of the laser fiber. The probe assembly may further include a depth stop configured to adjust an insertion length of the probe portion. The probe assembly is configured for emitting laser energy without creation of hot spots.

In certain implementations, the diameter of the probe portion is about 1.1 mm to about 1.65 mm, about 1.15 mm to about 1.6 mm, about 1.2 mm to about 1.55 mm, about 1.25 mm to about 1.5 mm, and/or about 1.3 mm to about 1.45 mm.

In various implementations, the probe portion may be configured to emit optical energy with a power output of about 4 watts, about 6 watts, about 8 watts, about 10 watts, or about 12 watts without causing tissue damage outside a target tissue region.

In some implementations, the probe assembly may be configured to ablate lesions having a cross-sectional diameter of about 8 mm to about 26 mm for a single probe trajectory along a probe eye view. Optionally, the probe assembly may be configured to ablate lesions having a cross-sectional diameter of about 8 mm to about 10 mm for a single probe trajectory along a probe eye view.

The probe assembly may further include a controller for controlling the temperature of the probe assembly using a proportional control algorithm. Such an algorithm applies a command signal to the controller proportional to the error between the current probe temperature and the desired probe temperature to be controlled. When a large difference in temperature exists, the command is larger thereby allowing the controller to correct the temperature difference more quickly. As the difference between the current probe temperature and desired temperature reduces, the command signal is smaller thereby allowing for a smaller correction to the cooling controller. Optionally, the controller may be configured to control the temperature of the probe portion using the proportional control algorithm to enable optical energy emission for up to about 15 minutes without causing tissue damage outside a target tissue region.

In some implementations, the depth stop may be a floating depth stop configured to be variably positioned along a length of a probe shaft for adjusting the insertion length of the probe portion within a tissue.

In various implementations, the diffuser element may be configured to evenly radiate diffused optical energy along an entire length of the diffuser element. Additionally, the diffuser element may be integrally formed at the distal tip of the laser fiber.

In various implementations, the probe portion may include a shaft bonded to a lens. The shaft may include a shaft lumen for receiving the laser fiber and a cryotherapy generating element that is configured for cooling tissue in proximity of the diffused tip element. The lens may form an enclosed chamber for receiving a distal end of the laser fiber, the diffuser element, and a distal end of the cryotherapy generating element. Optionally, a distal end of the cryotherapy generating element is proximally offset from a distal end of the laser fiber by a distance of about 0.1-1 mm. Additionally and/or alternatively, the shaft may also include a temperature sensor configured to measure temperature of a distal section of the probe portion. Optionally, the temperature sensor may be proximally offset from a distal end of the laser fiber by a distance of about 1.5 mm to about 3.5 mm. In various embodiments, the lens may include a glass capsule. Additionally and/or alternatively, the shaft may be a doubled walled tube comprising an inner tube and an outer tube. Optionally, the shaft may be bonded to the lens by reflowing the outer tube over at least a portion of the lens.

In various implementations, the temperature modulated probe assembly may also include a heat shrink included within the shaft and configured to hold a distal end of the cryotherapy generating element at a stable position with respect to a distal end of the laser fiber.

In various implementations, the temperature modulated probe assembly may also include a cryotherapy generating element including a cooling tube having an internal diameter of about 0.250 to 0.350 mm and an outer diameter of about 0.400 to 0.650 mm.

In various implementations, the temperature modulated probe assembly may also include an umbilical sheath for transmitting inputs or outputs between the probe portion and a plurality of probe connectors or the between the probe portion and a control unit.

In various implementations, the probe portion may include gradations for indicating the insertion length of the probe within the tissue.

### BRIEF DESCRIPTION OF THE DRAWINGS

FIGS. 1A and 1B illustrate example components of an example probe apparatus.

FIG. 1C illustrates a close-up view of an example variable length probe.

FIGS. 1D and 1E illustrate cross sectional views of an example probe showing example components included therein.

FIG. 2 illustrates an image of the ablation region obtained using the probe of the current disclosure.

FIG. 3 illustrates a schematic representation of example of hardware included in various components of the systems of this disclosure.

### BRIEF DESCRIPTION OF DISCLOSED EMBODIMENTS

The devices and methods of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, proximal, distal, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. It should be noted that the terms “proximal” and “distal” as used herein, are intended to refer to a direction toward (proximal) and away from (distal) a surgeon or other user of the device.

Measurements and ranges described herein relate to exemplary implementations and can identify a value or values within a range of 1%, 2%, 3%, 4%, 5%, or, preferably, 1.5% of the specified value(s) in some implementations.

Stereotactic laser interstitial thermal therapy (LITT) is a minimally invasive and cytoreductive neurosurgical technique for a variety of central nervous system (CNS) lesions ranging from tumors to epilepsy foci. During a LITT ablation, light energy emitted by the laser is converted into thermal energy by the surrounding tissue. This process occurs when photons emitted by the laser optical fiber are absorbed by tumor cell chromophores, resulting in chromophore excitation followed by release of thermal energy.

The surgical workflow for performing LITT includes imaging the target tissue (e.g., using Magnetic resonance imaging (MRI), CT scan, etc.) for use in determining a probe trajectory (or trajectories) for ablation using computer-based stereotactic neuro-navigation system, following the first-order principle of treating the lesion down the center of its long axis. A fiberoptic laser probe is then inserted into the target tissue (e.g., via a burr hole) along the determined trajectory. Once the probe is secured in place, real-time intraoperative imaging is performed of the patient with the probe in place. After initial MRI images are captured to verify probe position, the ablation process is initiated. During this time, firing of the laser probe is interleaved with periodic acquisition intraoperative MRI images. The MRI images are used for verification of the initial probe position and of any new probe position that results from advancing or withdrawing the probe, and measurement of tissue temperature using MR thermometry. Utilizing MRI imaging in real time guidance may provide controlled accuracy, while contemporaneous thermography may provide accurate temperature information in determining whether a tissue has been ablated or necrotized. Examples of such workflows are described in, for example, U.S. Pat. No. 10,327,830 entitled “Cryotherapy, thermal therapy, temperature modulation therapy, and probe apparatus therefor,” U.S. Pat. No. 10,188,462 entitled “Image-guided therapy of a tissue,” U.S. Pat. No. 9,387,042 entitled “Hyperthermia treatment and probe therefor,” U.S. Pat. No. 9,333,038 entitled “Hyperthermia treatment and probe therefor,” and U.S. Pat. No. 8,979,871 entitled “Hyperthermia treatment and probe therefor,” the contents of each of which are incorporated herein by reference in its entirety.

In one aspect, the present disclosure relates to reduced profile probe designs. Reducing the profile of a probe is desirable for achieving minimally invasive surgery, performing surgical operations upon small bodies such as infants, juveniles and animals, and reaching otherwise difficult-to-reach in situ locations without negatively impacting surrounding tissues. A reduced profile probe, for example, can allow entry into small and narrow spaces in the brain while reducing patient injury. A low profile probe may include multiple internal lumens. Low profile probes may be configured with selected materials, lumen structures, and layer structures to provide desired and/or selected mechanical properties including straightness, rigidity, torque strength, column strength, tensile strength, kink resistance, and thermal properties such as thermal stability and thermal stress capacity. Currently, the smallest known probe diameter for LITT therapy is about 1.7 mm and which achieves a maximum ablation size of about 20 mm. The aforementioned diameter restricts the capability of the probe to ablate certain types of lesions (e.g., deep seated lesions, small size lesions, pediatric patient lesions, lesions that are in certain critical parts of the brain, etc.). For example, treatment of Hypothalamic Hamartoma (present deep seated and small tumors), Corpus Callosotomy (curved structure requiring multiple trajectories), and Tubular Sclerosis (requires multiple trajectories to treat multiple tubers) will benefit from a smaller diameter probe. However, reducing the diameter without compromising on the thermal or energy output of the probe creates several technical challenges. For example, a small diameter probe lens for a smaller diameter probe will have a smaller surface area and thus a higher irradiance (“irradiance”=energy density) leading to the creation of “hot spots” in tissue surrounding the target region resulting in over-heating, damage, and phase change (in MRI imaging) of surrounding tissue. Furthermore, the probe lens will need to be configured to withstand the higher irradiance without shattering or damage. Reduction in the probe diameter can also compromise the cooling performance because of the higher energy density and/or the reduction in volume of the cooling fluid available within the probe shaft (due to reduction in the diameter of the cooling fluid tube). It should be noted that the configuration of a smaller diameter probe described herein is not merely a design choice but a solution to a technical problem. Configuring a smaller diameter probe that does not compromise on the energy output or the cooling performance presents to persons of ordinary skill in the art with a real world problem.

The current disclosure describes a smaller diameter probe (i.e., smaller than about 1.65 mm or about 1.70 mm) that does not compromise on the energy output or the cooling performance, while avoiding the higher energy density and is configured to solve significant design challenges. The disclosure describes a probe having a diameter of about 1.1 mm to about 1.65 mm, about 1.15 mm to about 1.6 mm, about 1.2 mm to about 1.55 mm, about 1.25 mm to about 1.5 mm, about 1.3 mm to about 1.45 mm, or the like. The diameter of the probe may be about 1.5 mm, 1.55 mm, 1.6 mm, or 1.65 mm. Furthermore, the probe is configured to emit even, controlled, and consistent energy allowing for higher power settings for ablating larger lesions as well. Furthermore, the probe's unique design, in combination with the disclosed system utilizes various power settings to achieve a wide range of ablation sizes (even greater than 20 mm). The disclosure thus provides a robust cooled probe design that is capable of surviving the power and ablation duration needed to achieve the ablation of lesion sizes of up to about 25 mm cross sectional length when seen from a probe-eye view in a single prove trajectory along a single probe trajectory. The cooled probe design is also capable of ablating smaller lesion sizes of as little as about 7-8 mm cross sectional length when seen from a probe-eye view along a single probe trajectory. As such, the cooled probe design of the current disclosure can be used to achieve a wide range of ablation sizes from about 8 mm to about 26 mm, about 10 mm to about 24 mm, about 12 mm to about 22 mm, about 14 mm to about 20 mm, about 16 mm to about 18 mm, about 20 mm to about 25 mm, about 8 mm, about 10 mm, about 24 mm, about 26 mm, or the like, along a single trajectory (i.e., cross section length of the ablation area when seen from a probe eye view).

For example, the disclosure describes a system for providing wide range of power settings for energy delivery (e.g., 4, 6, 8, 10, 12 watts, and up to about 20 watts) utilizing a suitable wavelength selected from about 800 nm to about 1100 nm (e.g., about 800 nm, about 900 nm, about 1000, about 1064 nm, or the like). The importance of the lower and upper power settings is that they enable more control at 6 watts to ablate smaller lesions and more power at 12 watts to ablate large lesions (at least 20 mm)—all in a small diameter probe. The disclosure also describes the use of a full fire diffused tip probe design that includes a lens capsule over the distal end of laser fiber (and diffuser element) and a controlled cooling algorithm that enables longer laser-firing (up to about 5 mins, about 1-5 mins, about 2-4 mins, about 3 mins, about 3-5 mins, about 5-10 mins, about 8-10 mins, about 10-15 mins, up to about 5 mins, up to about 10 mins, up to about 15 mins, or the like).

The disclosed probe also provides a single-length design offering users one probe that can be adjusted for varying lesion depths and locations and reduces hospital inventory. Specifically, the present disclosure relates to a variable length probe apparatus having a variable length probe structure including a probe and an adjustable depth stop to facilitate access to both shallow and deep targeted tissue areas. The variable length probe apparatus may be configured to accommodate lesions located at varying depths by repositioning over the adjustable depth stop.

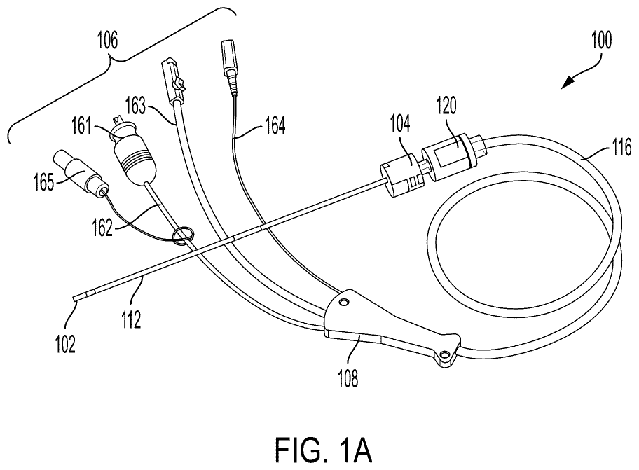

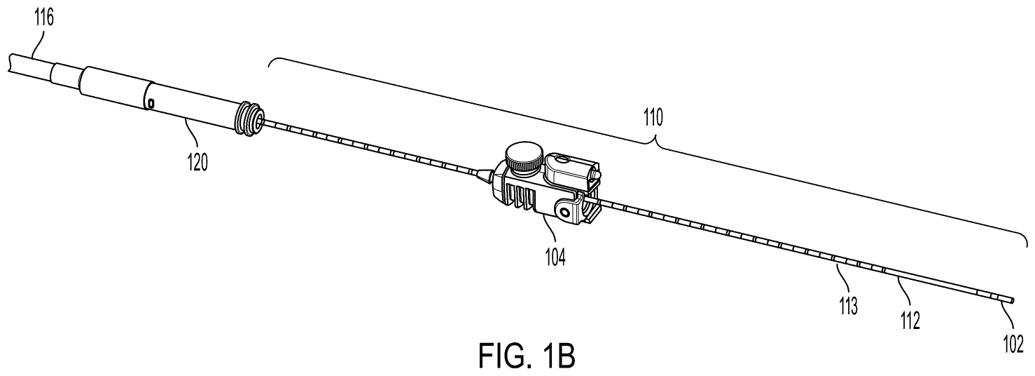

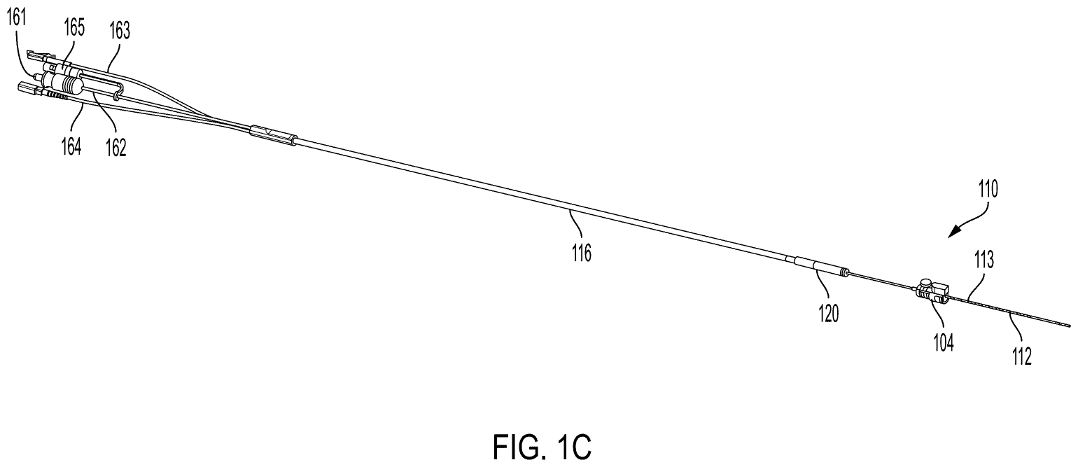

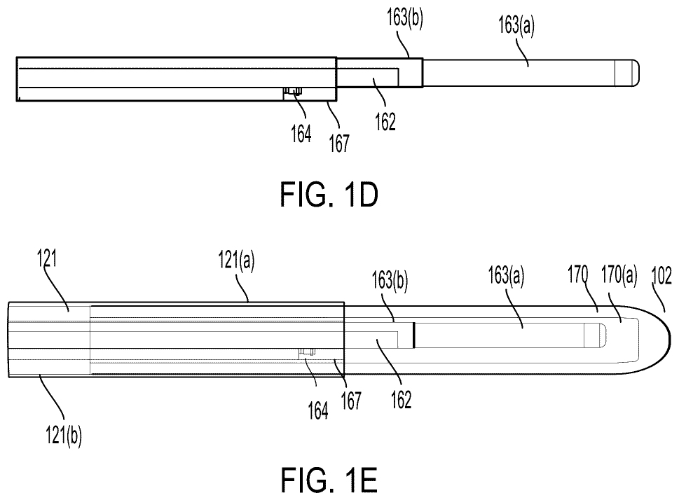

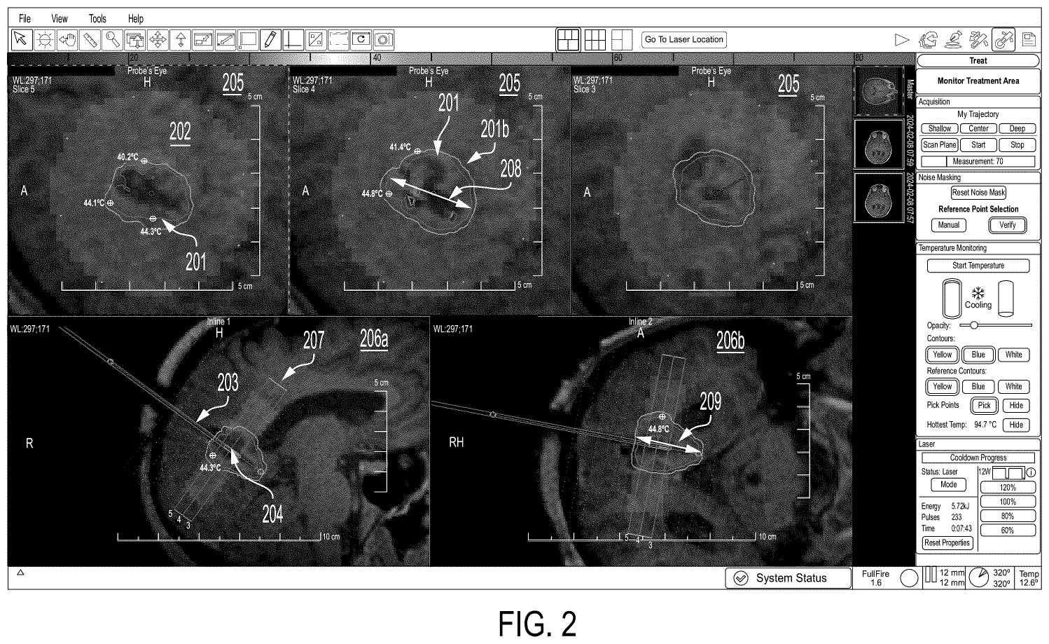

Referring now to FIGS. 1A and 1B, an example probe apparatus 100 is illustrated. As shown in FIGS. 1A and 1B, a probe tip 102 indicates an insertion end of the probe 112. An end opposite the insertion end is coupled or connected to (via an umbilical sheath 116) probe connectors 106 for energy delivery, cooling, etc. extending from a probe interface boot 108 (e.g., a probe hub). Examples of the probe connectors 106 can include, without limitation, a cooling tube connector cap 161 coupled to a proximal end of a cooling tube 162, a data collection interface (not shown here) coupled to a proximal end of a temperature sensor (e.g., a fiber optic temperature sensor (FOTS)) 164, an energy delivery device (e.g., a laser) (not shown here) coupled to a proximal end of a fiber optic assembly 163 (e.g., a DTP fiber assembly), and a probe detection dongle 165.

Between the probe 112 and the boot 108 is a flexible umbilical sheath 116 for carrying inputs and outputs (e.g., energy, control signals, cooling gas or fluid, and/or heating gas or fluid) between the probe 112 and the probe connectors and/or between the probe 112 and a control unit (not illustrated). A transitional probe interface 120 may be included between the flexible umbilical sheath 116 and the probe 112 that is configured for case of grasping and manipulation of the probe 112 when positioning the probe 112 in a target tissue region. The probe interface 120 also provides an interface for cabling alignment of the probe assembly 110 with a probe driver (not shown here). The probe interface 120 may include one or more indentations (not shown here) for grasping the probe interface 120 upon positioning or otherwise manipulating the probe 112. Further, the probe interface 120 may include one or more vents 138 for venting return gasses in a gas-heated or fluid-cooled probe.

In various embodiments, the flexible umbilical sheath 116 may include an inner lumen. The lumen may be used for carrying inputs and outputs (e.g., energy, control signals, temperature sensor optical fibers, a deflection wire, cooling gas or fluid, and/or heating gas or fluid) between the probe 112 and the probe connectors and/or between the probe 112 and a control unit (not illustrated). In some examples, the inner lumen may include one or more tubes, lumens, or other divisions to separate various inputs and outputs directed through the lumen. Optionally, the lumen may include windings on the inner surface of the umbilical sheath in order to protect the components within and/or may be configured to supply a particular kink resistance value, torque strength, and/or tensile strength to the flexible umbilical sheath 116. For example, the lumen may include a number of lumens or tubes to enable cooling fluid supply to and cooling fluid return from a fluid-cooled probe. In one embodiment, the coil structure resulted in optimal kink resistance and a tighter coil (as compared with a looser coil) resulted in better column strength. In another embodiment, one or more of the multiple layers has a braided structure. The flexible umbilical sheath 116 may be designed using non-ferro-magnetic materials for MRI compatibility. For example, the windings may be composed of a polymer or poly-vinyl material. In other examples, the flexible umbilical sheath may be composed of PTFE, PEEK, Polyimide, Polyamide and/or PEBAX, and the winding material may include stainless steel, Nitinol, Nylon, and/or PEEK. The materials of the windings and/or the external covering of the flexible umbilical sheath 116 may be selected in part for thermal stability. In the example of a thermal therapy probe cryotherapy probe, or a temperature modulated probe, the materials of the windings and/or the external covering of the flexible umbilical sheath 116 may be selected to withstand extremely hot and cold temperatures without incurring damage during flexing.

FIG. 1A illustrates the depth stop adjustment 104 being positioned to expose almost the entire length of the probe 112, while FIGS. 1B and 1C illustrate depth stop adjustment 104 being positioned to expose less than the entire length of the probe 112 (i.e., the probe 112 with the depth stop 104 forming a variable length probe assembly 110). A variable length probe assembly 110 has a variable length probe structure including a probe 112 and an adjustable depth stop 104 to facilitate access to both shallow and deep targeted tissue areas; that is, the same probe 112 can accommodate lesions located at varying depths by repositioning over the adjustable depth stop 104.

As illustrated in FIGS. 1B and 1C, an enlarged view of the probe 112 and the adjustable depth stop 104 demonstrates measured gradations 113 (e.g., centimeters, millimeters, inches, etc.) printed along the shaft of the probe 112. An operator may align the measured gradations with the adjustable depth stop 104, for example, by sliding a position of the adjustable depth stop 104 along the shaft of the probe 112 until the desired depth measurement aligns with the probe end of the adjustable depth stop 104. In another example, the operator may align the desired depth measurement with the sheath end of the probe 112 (e.g., for clearer visibility). In this example, the gradations may be applied to the shaft of the probe 112 to compensate for a length of the adjustable depth stop 104. In a further embodiment (not illustrated), the adjustable depth stop 104 may include a window (e.g., cut-out portion, clear portion, or clear, magnified portion) for aligning a desired depth measurement while slidably positioning the adjustable depth stop 104. In this example, the gradations may be applied to the shaft of the probe 112 to compensate for the portion of the length of the adjustable depth stop 104 from the probe end to the depth selection window.

Instead or in addition to the printed gradations, in other embodiments, the shaft of the probe 112 may include a series of mating points for mating with the adjustable depth stop 104. For example, the shaft of the probe 112 may include a series of bumps (e.g., every 5 millimeters, 10 millimeters, etc.) and the adjustable depth stop 104 may include one or more mating depressions for engaging with at least one of the series of bumps. The mating points, for example, may be used to more precisely align the adjustable depth stop 104 with a particular depth setting.

Using the adjustable depth stop 104 with the guidance of the printed gradations upon the shaft of the probe 112, an operator may modify the probe length in situ. For example, during a patient operation, after applying a procedure at a first selected depth, an operator may vary the length or depth relatively rapidly to a second selected depth. To allow for varying the probe length of the probe 112 in-situ while controlling the internal deployment of the probe tip, the shaft of the probe 112 may be configured with a selected kink resistance value, in addition to column strength and/or torque strength and/or tensile strength and/or thermal stability. The probe-side end of the adjustable depth stop 104, in some examples, may be designed to fit over and/or mate with a minibolt mounted to the skull of a patient or a probe driver (as described in U.S. patent application Ser. No. 17/661,241).

### Depth Stop:

The depth stop may be a floating depth stop (FDS) lock that includes two symmetrical clamping or “locking blocks” placed on either side of the probe shaft, and is configured to be variably positioned along a length of a probe shaft to control the length of the probe inserted within a tissue. Each of the locking blocks has a channel that closely matches the shape of a probe shaft (e.g., shaft of probe 112), but has a slightly smaller cross section area compared to the probe shaft outer diameter. A thumb screw may be tightened which forces the two locking blocks close to each other, clamping the probe shaft and preventing it from moving. The interference between the locking blocks, and probe shaft is tightly controlled (by controlling the size of the locking blocks with respect to the probe shaft diameter) which ensures a very consistent lock. Also, the screw cannot be “over tightened” and damage the probe shaft because it is protected by the rigid locking blocks pressing together. In some embodiments, the adjustable depth stop can be used with the probe which inserted into a cranial cavity using a cranial access assembly as described in PCT Patent Publication number PCT/US23/78536 filed Dec. 20, 2023 that claims priority to U.S. provisional patent appl. No. 63/382,092 filed Nov. 2, 2022 (i.e., the depth stop is configured to interface with the cranial access assembly), the disclosure of which is incorporated in here in its entirety. Other examples of depth stops are within the scope of this disclosure such as those described in U.S. Pat. No. 10,327,830, the disclosure of which is incorporated in here in its entirety.

Referring now to FIGS. 1D and 1E, a cross-section view of probe 112 is illustrated. As shown in FIG. 1E, the probe 112 may include a probe shaft 121 (e.g., a shaft extending from the probe interface 120) that provides one or more lumens for receiving (and/or operably coupling to) portions of the thermal therapy generating element (e.g., fiber optic assembly 163), the cooling-therapy generating element (e.g., cooling tube 162), and a temperature senor (e.g., 164) extending from the probe interface 120. The probe shaft 121 is coupled to (as discussed below) a lens capsule or lens 170 to provide a sealed chamber 170 (a) that includes distal portions of the fiber optic assembly 163, the cooling-therapy generating element and/or the temperature sensor. The distal ends may be included in a single shaft lumen and/or within multiple lumens provided within the shaft 121. FIG. 1D illustrates the cross-section view of the probe 112 without the probe shaft 121.

In some embodiments, the shaft 121 of the probe 112 may be composed of one or more biocompatible materials selected at least in part for flexibility of the shaft portion such that the shaft portion may bend away from the skull. The material may be chosen to provide a desired flexibility so as to minimize translation of side loads to the lens (leading to lens fracture) during insertion. Examples of shaft material may include, without limitation, PEEK, pultruded tube, fiberglass, multi-layered material (e.g., including polyimide, PEED braid, polyimide layers). Optionally, at least a portion of the shaft may be braided, may include windings, may include patterned holes, etc. in order to provide the desired flexibility.

Optionally, the shaft may be designed to have varying rigidity or flexibility along its length. Shaft materials may include, in one example, polyimide for rigidity in a first shaft portion designed for interstitial deployment, and PTFE for flexibility in a second shaft portion interfacing with the umbilical sheath. Additionally, the shaft may be composed of multiple layers of material, such as polyimide under an etched layer of PTFE, to provide for better bonding characteristics at the interface between the shaft and the lens (as discussed below).

The fiber optic assembly 163 may extend from an optical energy delivery device (e.g., laser) and may include a laser fiber or fiber 163 (b) (e.g., a fiber optic laser fiber) configured for transmitting optical energy from the optical energy generating device along the length of the fiber to the tip of the fiber. In some example, embodiments, the fiber 163 (b) may include, without limitation a core (about ˜375-425 μm, about 390-400 μm, about 400 μm, or the like), a clad (about ˜ 420-450 μm, about 425-445 μm, about, 430 μm, about 440 μm, about 450 μm, or the like), a hard clad (about ˜ 460-480 μm, about 465-475 μm, about 470 μm, about 460 μm, about 480 μm, or the like), a jacket (about ˜ 550-650 μm, about 575-625 μm, about 550 μm, about 600 μm, about 650 μm, or the like), and a connector. The fiber optic assembly 163 also includes a diffuser element 163 (a) attached or coupled to, and/or integrally formed with, the distal tip of the laser fiber 163 (b) and is configured to receive the transmitted optical energy and provide a diffused energy output (i.e., scramble the light wavefronts and reduce its spatial coherence). This helps reduce the irradiance by emitting diffused energy in order to eliminate creation of “hot spots” and to reduce chances of over-heating of tissue and causing phase change (“blue pixels”). The diffused energy output may be configured to be evenly diffused in all radial directions along the length of the diffuser element 163 (a) to form a full-fire probe. Alternatively, the diffused energy output may be configured to be unevenly diffused. The diffuser element may be any now or hereafter known optical diffuser such as, without limitation, ground glass optical diffusers, engineered optical diffusers, hexagonal light mixing rods, diffuse reflectors, holographic diffusers, or the like. Optionally, the diffuser element 163 (a) may be uncladded and/or etched. In various implementations, the diffused energy emission and even energy output (with or without the cooling therapy) also allows for longer laser emission times (up to about 10 mins) to deliver up to about 30 W of power without over-heating or phase changes. The diffuser element 163 (a) may have a length of about 4-8 mm, about 5-7 mm, about 6 mm, or the like.

The cooling-therapy generating element (e.g., cooling tube 162) may extend into the shaft 121 from, for example, a cooling tube connector cap 161 (cap being configured to attach to a cooling fluid reservoir) via the cooling tube connector cap 161. The cooling tube 162 is configured to deliver cooling fluid at a desired pressure/temperature within the chamber 170 (a) for cooling the probe and/or the surrounding tissue to a desired temperature (as discussed below).

A temperature sensor 164 may be provided within the shaft and may be communicatively coupled to a data collection interface. The temperature sensor 164 is configured to collect temperature data of the probe (e.g., within the chamber 170 (a)).

Further, as shown in FIG. 1E, a lens 170 (e.g., a clear capsule) may be provided for protecting the diffuser element 163 (a) and one or more of the other components within the shaft 121. Specifically, the diffuser element 163 (a) may be enclosed within and protected by a capsule or lens 170. Optionally, the lens 170 may also enclose at least a portion of the laser fiber 163 (b) as shown in FIG. 1E for protecting the fiber and/or for preventing loss of optical energy from the distal tip of the fiber 163 (b). Additionally, the lens 170 may also enclose at least a portion of the cooling tube 162 for providing an enclosed space or chamber 170 (a) within the probe distal section where the distal ends of the laser fiber 163 (b) and the cooling tube 162 are received for providing an optimal temperature control of the distal tip of the probe (and/or surrounding tissue). The lens may further extend over at least a portion of the heat shrink 167 and enclose the temperature sensor 164. The lens may be made from a material that is transparent at the operating wavelength of the fiber optic (e.g., at 1064 nm), that can withstand thermal stresses and high temperatures (corresponding to the cooling performance and energy delivery within the enclosed space 170 (a) formed by the lens 170), and that can withstand handling forces without breaking. Examples of materials may include, without limitation, sapphire (machined sapphire, grown and capped sapphire, etc.), aluminum oxynitride, glass (e.g., borosilicate, fused silica glass, etc.), plastic (e.g., Ultem, polycarbonate, etc.), and/or combinations thereof.

In some embodiments, the lens 170 may be attached to the shaft 121 (e.g., by bonding) to extend within and/or cover at least a portion of the shaft. Optionally, the lens 170 may be attached to the shaft 121 at the distal end of the shaft. In order to achieve a smaller diameter probe of the current disclosure, the probe shaft and lens diameter must be reduced. This diameter reduction can often complicate the process of bonding or connecting the shaft 121 to the lens 170. However, achieving a properly sealed joint between the lens and the shaft is critical in that it must properly align the lens to the probe shaft and also retain internal cooling gas pressure without leaking.

As such, in some implementations, the shaft 121 may include a dual tube design (as shown in FIG. 1E), and a sealed joint may be created by reflowing the outer tube 121 (a) of the dual tube shaft over the lens 170 as shown in FIG. 1E. Such a dual tube design maximizes stiffness of the joint as well as the probe shaft. An inner tube 121 (b) is used to add stiffness and terminates at the joint to the lens 170, while a thin-walled outer tube 121 (a) slides over the inner tube and overlap the lens forming a joint solidified by the “reflow process”. As such, a “reflow” or over molding process may be used for connecting the shaft to the lens without the use of any bonding agent. In some embodiments, the process melts and molds a suitable outer-tube material (e.g., PEEK) around the lens to form a thin-walled outer tube that overlaps a proximal portion of the lens 170. The material is selected melts at a high temperature, and due to the short overlap distance and dissimilarity between materials of the shaft and the lens, a successful bonding is created using the reflow process (with careful control of multiple variables such as, without limitation, temperature, heating area, heating time, and cooling time).

In order to maximize the amount of heat energy which can be applied through the fiber and thereby to effect treatment of larger tumors, it is highly desirable to effect cooling of the tissue immediately surrounding the end of the fiber so as to avoid overheating that portion of the tissue. Overheating beyond the coagulation temperature is unacceptable since it will cause carbonization which will inhibit further transmission of the heat energy. Thus without the cooling it is generally necessary to limit the amount of heat energy which is applied. As energy dissipates within the tissue, such a limitation in the rate of application of energy limits the size of the tumor to be treated since dissipation of energy prevents the outside portions of the tumor from being heated to the required coagulation temperature.

The cryotherapy-generating element (e.g., the cooling tube 162) includes, for example, a cooling fluid or gas, for supplying cryotherapy to the effected tissue. In some examples, the cryotherapy-generating element may include a flow of fluid such as gaseous carbon dioxide, liquid nitrogen, or liquid argon. As described in, for example, U.S. Pat. Nos. 7,691,100 (the disclosure of which is included herein in its entirety); a cooling fluid is pumped through the cooling tube 162 and allowed to escape from its distal end around the diffuser end 163 (a) within the lens 170 of the fiber optic before being returned via a collection or return duct (not shown here). Optionally, the cross-section of the return duct may be about ten times the diameter of the cooling tube. Specifically, a suitable coolant (e.g., liquid nitrogen, carbon dioxide gas, etc.) is compressed and condensed at the supply end and is allowed to escape from distal end of the cooling tube to create a cooling zone.

As discussed above, reduction in the probe diameter can compromise the cooling performance because of the higher energy density (concentrated heating), the smaller surface area in contact with tissue and/or the reduction in volume of the cooling fluid available within the probe shaft (due to reduction in the diameter of the cooling tube). Specifically, the cooling tube 162 delivers cooling fluid to the probe tip 102 (i.e., within the chamber 170 (a) at or near the distal end of the probe) and the distal end of the cooling tube 162 within the probe is configured to be situated in an optimal position relative the distal end of the fiber 163 (b) and the temperature sensor 164 (a). The location of the distal end of the cooling tube relative the distal end of the laser fiber and the temperature sensor is determined based on careful and extensive experimentation and optimization in order to maximize cooling where the laser fiber delivers maximum energy into tissue while ensuring that the cooling tube cannot interfere with the functioning of the laser fiber itself nor be in a direct energy path. All of these relative locations are not simply scaled down from larger probe but demanded technical skill to optimize performance in a smaller design. In some embodiments, the distal end of the cooling tube is offset proximally from the distal end of the fiber 163 (b) by a distance of about 0 mm to about 1 mm, about 0.2-0.9 mm, about 0.3-0.8 mm, about 0.4-0.7 mm, about 0.5-0.6 mm, or the like. In some embodiments, sensor 164 is offset proximally from the distal end of the fiber 163 (b) by a distance of about 1.5 mm to about 3.5 mm, about 1.75-3.25 mm, about 2-3 mm, about 2.25-2.75 mm, or the like. In some embodiments, sensor 164 is offset proximally from the distal end of the cooling tube by a distance of about 0 mm to about 3.5 mm, about 0.5-3 mm, about 1.0-2.5 mm, about 1.5-2 mm, or the like. Optionally, in some embodiments, at least a portion of the cooling tube 162 and the laser fiber 163 (b) (and/or the temperature sensor 164) may be encased within in an ultra thin-wall clear heat shrink (167). The heat shrink forms a protective seal holding the components in respective desired positions (i.e., maintains the offsets between the various components), and is composed of a material that shrinks in diameter when heated, resulting in secure scaling.

In addition to the location of the distal end of the cooling tube, the cooling tube is also configured to ensure delivery of sufficient gas pressure and flow to the probe tip yet while being sized to be included in the smaller diameter probe. This is achieved by keeping the internal diameter of the cooling tube as large as possible (similar to the diameter of cooling tubes within larger diameter probes) while reducing the outer diameter of the cooling tube (i.e., a cooling tube with reduced wall thickness is provided). In various implementations, the internal diameter of the cooling tube is about 0.250 to about 0.350 mm, about 0.260 to about 0.340 mm, about 0.270 to about 0.330 mm, about 0.280 to about 0.320 mm, about 0.290 to about 0.310 mm, about 0.300 mm, about 0.290 mm, about 0.295 mm, about 0.305 mm, about 0.310 mm, about 0.315 mm, or the like. The outer diameter of the cooling tube may be about 0.400 mm to about 0.650 mm, about 0.450 mm to about 0.600 mm, about 0.500 mm to about 0.550 mm, or the like. The material of the cooling tube is selected such that the cooling tube can withstand the high pressure required for efficient over its long length despite the reduced wall thickness. Examples of such material can include, without limitation, polyimide, PEEK, glass or the like.

Temperature production of the probe 112, in some implementations, is refined based upon temperature measurements obtained by a temperature sensor (e.g., FOTS 164). The temperature sensor is positioned to be offset back from the cooling tube exit (i.e., the distal end of the cooling tube). This is to ensure the sensor in not in a direct path of high pressure gas expanding at the cooling tube exit into the lens 170 so as to avoid damage the sensor and/or to avoid inaccuracies in temperature sensing of the probe tip using the sensor. It should also be noted reduction of the probe diameter may require selection of a probe tip lens material (of lens 170) that can be manufactured in the required lower dimensions (e.g., glass), and the cooling performance is optimized for the selected material. Lower thermal conductivity of glass impacts cooling performance and can serve to limit thermal stress in the lens wall, and the cooling control algorithm is, therefore, adjusted and optimized to ensure adequate cooling at the probe tip. For efficient cooling of a probe tip (with a glass lens) of a smaller diameter probe that causes higher energy or heat density, the current disclosure also describes a novel cooling algorithm that effectively cools tissue next to the probe tip (and/or the probe tip itself) to avoid over-heating while deep penetration of energy (i.e., allow for more ablation power for a longer time (e.g., up to about 15 minutes) despite the smaller diameter of the probe). A controller may control, using the cooling algorithm, the probe temperature production by controlling the cryotherapy generating element. The proposed cooling algorithms is a proportional control algorithm that is used to optimize the reaction rate to disturbance of the probe tip temperature and to speed up the cooling rate at the beginning of a laser shot.

Typically for larger diameter probes, the laser is not enabled for therapy delivery until the probe tip has been cooled to desired temperature (e.g., about 11-14° C.). Specifically, the controller may be configured to disable triggering of the laser energy delivery until probe cooling has started (i.e., tissue ablation only occurs when cooling is active). When cooling starts, a target probe tip temperature is calculated as the middle of the target treatment range. Based on the target probe tip temperature, an initial pressure setpoint is determined and set in the cooling fluid (e.g., CO₂ gas) pressure controller. Once the cooling fluid delivery starts at the set pressure, the instantaneous probe temperature is constantly monitored (e.g., using the FOTS 164), and used to periodically update the pressure (e.g., every 100 milliseconds) using a cooling algorithm. However, a probe inserted into the brain is initially at ˜37° C. or body temperature. As such, when therapy needs to be delivered, a user must wait for the system to cool the probe tip to the set temperature range before the laser is enabled to deliver energy thereby causing delays before (and/or during) treatment sessions.

The proposed proportional control algorithm proportionately adjusts the cooling pressure needed for probe tip cooling based on the current difference between the current probe temperature and the target temperature. At the start of a laser shot, this error is maximum (e.g., when the probe is at 37° C. and the target range is 11-14° C.), and the proportional control algorithm delivers maximum cooling pressure based on this difference so as to cool the probe tip at a relatively fast rate. This reduces any delays a user might experience before the laser can be fired. As the probe cools, the difference between the current probe temperature and the target temperature reduces, and the proportional control algorithm reduces the pressure of the cooling fluid accordingly such that the rate of cooling is reduces. The proportional control algorithm also allows for faster and more efficient cooling and for attaining lower target temperature ranges (e.g., about, 5-10° C., about 0-5° C., about 11-14° C., or the like).

As discussed, the cooling algorithm dynamically adjusts pressure by adjusting the pressure setpoint based on the instantaneous probe tip temperature sensor reading compared to the target probe temperature. Specifically, the cooling algorithm is a proportional control algorithm in which the amount of cooling fluid pressure increased or decreased is proportional to the difference between the current temperature reading and the target temperature reading. A larger difference in temperature results in a larger pressure change, a smaller difference in temperature results in a smaller pressure change. The resulting new pressure setpoint is then clamped to ensure it remains in the range of 0 to 3200 kPa, and finally set in the pressure controller as the current pressure setpoint.

In various embodiments, a Probe Temperature Error is determined as the target temperature reading subtracted from the current temperature reading. And,

$$New Pressure Setpoint = ( ( Probe Temperature Error ) * Cooling Coefficient ) + Current Pressure Setpoint$$

Where, cooling coefficient ˜50 kPa per degree Celsius.

The proportional control algorithm allows for tighter control of temperature of the probe (via the cryotherapy generating element), and further aids in reduction of the diameter of the probe. It also allows the probe to achieve a multitude of power setting and/or longer periods of laser emission time. Optionally, PID control algorithms may also be used.

The cryotherapy-generating element, in some embodiments, is designed for compatibility with the thermal therapy-generating element. For example, the fluid or gas supplied by the cryotherapy-generating element may be selected to avoid interference with the transmission of heat energy by the thermal therapy-generating element (e.g., will not alter laser light attenuation due to absorption by the coolant fluid).

In some implementations, an energy output pattern of the probe includes simultaneous activation of at least one cryotherapy-generating element and at least one thermal therapy-generating element. For example, emissions of both a cryotherapy-generating element and a thermal therapy-generating element may be combined to refine control of temperature emission of the temperature modulation probe. Modulation may further be achieved by varying output of at least one cryotherapy-generating element relative to at least one thermal therapy-generating element. For example, laser power, pulse timing, RF cycling, HiFU element frequency and/or power, fluid or gas pressure, fluid or gas temperature, and/or flow rate may each be varied to obtain temperature modulating output and/or controlled temperature output.

In various embodiments, the resulting outer diameter of the probe 112 is about 1.1 mm to about 1.65 mm, about 1.15 mm to about 1.6 mm, about 1.2 mm to about 1.55 mm, about 1.25 mm to about 1.5 mm, about 1.3 mm to about 1.45 mm, or the like. The diameter of the probe may be about 1.5 mm, 1.55 mm, 1.6 mm, or 1.65 mm. The length of the probe 112 may be configured to be long enough to accommodate all use cases. For example, the length may be about 130 mm to about 180 mm, about 140 mm to about 170 mm, about 150 mm to about 160 mm. As discussed, the depth stop 104 allows for locking the insertion depth of the probe at any location along the length of the probe (i.e., from about 0 mm to about 180 mm, about 10 mm to about 170 mm, about 20 mm to about 160 mm, about 30 mm to about 150 mm, or the like).

FIG. 2 illustrates an image of, for example, a display (or a graphical user interface) of the ablation region obtained using the probe of the current disclosure (shown as 203 in FIG. 2). The display shows five (5) different anatomical views of an ablation region within a brain: three probe's eye views (205) perpendicular to the laser delivery probe at three slice levels 207, respectively; and two probe's inline views (206a and 206b) shown parallel to probe 203. The image represents a single time point during an ablation procedure where many such images are displayed to the user during energy delivery. The probe's eye views 205 are aligned to the tip of the probe tip 204 in order to capture heating during laser ablation illustrated by a color coded (or grey scale) tissue temperature overlay 202. The contour line 201 outlines the region of thermal damage representing irreversible cell death. An alternate contour line 201b may show an outer region of thermal damage that may be affected but not irreversibly damaged tissue. Ablation size can be measured by the cross-sectional dimension of the contour line 201 in its largest extent both in probes eye views 208 and the inline view 209. The ablation size can be small (about 5-10 mm) or may extend to be as large as about 20-30 mm (similar to the ablation sizes obtained using larger diameter probes using at the same power output) when evaluated for a single probe trajectory shown in FIG. 2. As such, the smaller size probes of the current disclosure allow for a larger range of ablation sizes compared to existing larger size probes, and provide the ability to achieve much smaller ablation sizes. Moreover, even larger ablation sizes can be achieved by adding more probe trajectories into the target region. The smaller diameter probe of the current disclosure can, therefore, be used to ablate deep seated lesions, small size lesions, pediatric patient lesions, lesions that are in certain critical parts of the brain, etc.

In further embodiments, rather than or in addition to the fiber optic assembly 163, the probe shaft may include other thermal-therapy generating elements such as, without limitation, one or more ultrasound elements and/or ultrasonic transducers capable of focal or diffuse heating using HiFU, microwave, RF, heating gas, heating fluid, or electrical heat thermal therapy-generating element, or the like. Each of the at least one thermal therapy-generating element may be configured to emit thermal energy in a side-firing, focal, or diffuse manner. In a particular example, a temperature modulation probe includes a circumferentially emitting thermal therapy-generating element. The temperature modulation probes of the present disclosure may be designed for insertion into a body cavity, insertion into vascular system, or interstitial deployment.

In various embodiments, the probe apparatus of the current disclosure may be inserted and/or secured to a skull using the cranial access assembly described in U.S. patent application Ser. No. 17/661,241 filed Apr. 28, 2022 (the disclosure of which is included herein in its entirety), U.S. Provisional Patent Application No. 62/382,092 filed Nov. 2, 2022 (the disclosure of which is included herein in its entirety), and/or any now or hereafter known cranial access assemblies.

The probe can be a laser delivery probe that is used to deliver laser interstitial thermal therapy. The probe is preferably composed of MR compatible materials allowing for simultaneous laser application and thermal imaging, and can be provided in multiple lengths and dimensions. Other types of probes that can be utilized with the components and procedures discussed herein include radiofrequency (RF), high-intensity focused ultrasound (HiFu), microwave, cryogenic, chemical release, which may include photodynamic therapy (PDT), and drug releasing probes. For example, modalities of probes other than a laser energy modality can be utilized.

Further probes can include temperature modulation probes including at least one thermal therapy-generating element (e.g., RF, HiFu, microwave, laser, electrical heat, heating fluid or supercritical fluid, heating gas, etc.) and at least one cryotherapy-generating element (e.g., cooling gas, cooling fluid, etc.). Each of the at least one thermal therapy-generating element and the at least one cryotherapy-generating element may be configured to emit respective thermal or cryo energy in a diffused manner. In a particular example, a temperature modulation probe includes a circumferentially emitting thermal therapy-generating element and a circumferentially emitting cryotherapy-generating element. The temperature modulation probes of the present disclosure may be designed for insertion into a body cavity, insertion into vascular system, or interstitial deployment.

Treatments in accordance with the descriptions provided in this disclosure include treatments that ablate (i.e., “treat”) a tissue to destroy, inhibit and/or stop one or more or all biological functions of the tissue. Ablation agents include, but are not limited to, laser, RF, HiFu, microwave, cryogenic, PDT and drug or chemical release. A corresponding probe and/or another instrument, such as a needle, fiber or intravenous line can be utilized to effect treatment by one or more of these ablation agents. A corresponding probe and/or another instrument, such as a needle, fiber or intravenous line can be utilized to effect treatment by one or more of these ablation agents. Treatments in accordance with the descriptions provided in this disclosure include treatments that create temporary or permanent physical-biological effects to tissue including freezing freeze-thawing, hyperthermia, coagulation, and/or vaporization of tissues. The temporary or permanent physical-biological effects can include alterations in biological function of cells, tissues, and/or body fluids. In a particular example, the treatment may cause the cells, tissues, and/or body fluids to be more receptive or sensitive to additional therapies or manipulations such as, in some examples, radiation therapy or chemotherapy. In a further example, the treatment may cause hemostasis, reduction or dissolution of thrombi or emboli, alteration of functional membranes including the blood-brain barrier, and/or renal filtration. The physical-biological effects may be caused directly by temperature change to the cells, tissues, and/or body fluids or indirectly (e.g., downstream) from the temperature change, such as alterations in heat shock proteins or immune reaction or status.

According to one embodiment, the probe apparatus described above can be used in conjunction with a robotic probe driver. The robotic probe driver can align and position a tip of the probe at a certain distance from a target area (e.g., target tissues in the brain) that is to be treated, via the cranial access assembly. The probe can be used to treat various brain diseases by using thermal ablation. The diseases can range from tumors to epilepsy. According to an embodiment, the probe is aligned to the target tissue and inserted into the brain until the tip reaches the target tissue. Thereafter, laser energy is transmitted through the probe and emitted from the tip inside the target area. The energy heats the tissues causing cell death. It must be appreciated that the temperature of the probe tip can be controlled using a cooling gas and thermal monitoring.

A variety of other agents and compositions comprising such agents can be delivered using the probe apparatus, including but not limited to chemotherapeutic agents, agents for treatment of neurodegenerative disease (e.g., neurotrophic factors or neuroprotective agents), antiepileptic agents, antidepressant agents, antipsychotic agents, anti-inflammatory agents, antifibrotic agents, antianxiolytics and the like.

In some embodiments, the devices of the present invention may operate in conjunction with a computer platform system, such as a local or remote executable software platform, or as a hosted internet or network program or portal. In certain embodiments, portions of the system may be computer operated, or in other embodiments, the entire system may be computer operated. As contemplated herein, any computing device as would be understood by those skilled in the art may be used with the system, including desktop or mobile devices, laptops, desktops, tablets, smartphones or other wireless digital/cellular phones, televisions or other thin client devices as would be understood by those skilled in the art.

The computer platform is fully capable of sending and interpreting device emissions signals as described herein throughout. For example, the computer platform can be configured to control emissions parameters such as frequency, intensity, amplitude, period, wavelength, pulsing, and the like, depending on the emissions type. The computer platform can be configured to record received emissions signals, and subsequently interpret the emissions. For example, the computer platform may be configured to interpret the emissions as images and subsequently transmit the images to a digital display. The computer platform may further perform automated calculations based on the received emissions to output data such as density, distance, temperature, composition, imaging, and the like, depending on the type of emissions received. The computer platform may further provide a means to communicate the received emissions and data outputs, such as by projecting one or more static and moving images on a screen, emitting one or more auditory signals, presenting one or more digital readouts, providing one or more light indicators, providing one or more tactile responses (such as vibrations), and the like. In some embodiments, the computer platform communicates received emissions signals and data outputs in real time, such that an operator may adjust the use of the device in response to the real time communication.

In some embodiments, the computer platform is integrated into the devices of the present invention. For example, in some embodiments, at least one component of the computer platform described elsewhere herein is incorporated into a probe assembly of the present invention, such as emissions parameter controlling means, emissions recording and interpretation means, communication means for the received emissions and data outputs, and one or more features for displaying the received emissions, data, and images. Probe assemblies having at least one integrated computer platform component may be operable as a self-contained unit, such that additional computer platform components apart from the device itself are not necessary. Self-contained units provide a convenient means of using the devices of the present invention by performing a plurality of functions related to the devices. Self-contained units may be swappable and disposable, improving portability and decreasing the risk of contamination.

The computer operable component(s) may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a manager, expert, researcher, or other professional of the particular results. Further embodiments of such mechanisms are described elsewhere herein or may standard systems understood by those skilled in the art.

### Surgical Kits

The disclosure also includes a kit comprising components useful within the methods of the disclosure and instructional material that describes, for instance, the method of using the probe assembly as described elsewhere herein. The kit may comprise components and materials useful for performing the methods of the disclosure. For instance, the kit may comprise a cranial bolt, a fixation device, a depth stop, a fiducial marker, probe(s) of the current disclosure and/or one or more trajectory adjustment guides. In other embodiments, the kit may further comprise software and electronic equipment. The software and electronic equipment may be presented in a compact form for portable use.

In certain embodiments, the kit comprises instructional material. Instructional material may include a publication, a recording, a diagram, or any other medium of expression which can be used to communicate the usefulness of the device described herein. The instructional material of the kit of the disclosure may, for example, be affixed to a package which contains one or more instruments which may be necessary for the desired procedure. Alternatively, the instructional material may be shipped separately from the package, or may be accessible electronically via a communications network, such as the Internet.

In one embodiment, the disclosure includes a kit for portable use. To facilitate portable use, a kit of the present disclosure may further include a razor or clipper for removing hair from a subject, a ruler or tape measure for measuring the location of a site for incision, a surgical marker or other implement for marking the site of incision, skin preparation material (i.e., antiseptic, alcohol pads) to clean the site of incision, a scalpel to perform the incision, a drilling instrument to perforate any bone, and any additional surgical and medical elements that may be useful for such an operation, such as surgical tape, gauze, bandages, surgical thread and needle, and the like.

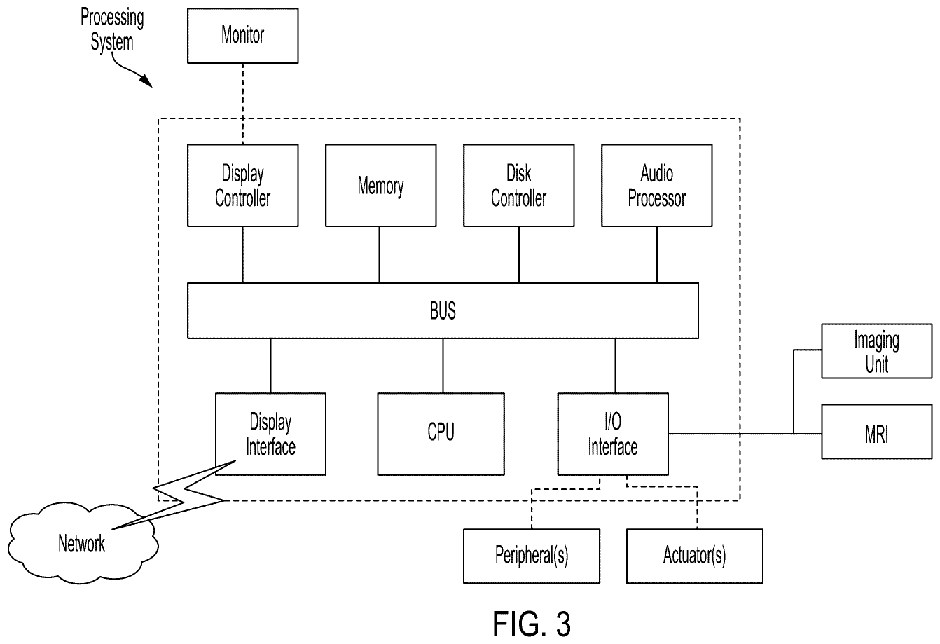

FIG. 3 illustrates an exemplary processing system, and illustrates exemplary hardware found in a controller or computing system (such as a personal computer, i.e., a laptop or desktop computer, which can embody a workstation according to this disclosure) for implementing and/or executing the processes, algorithms and/or methods described in this disclosure. A processing system in accordance with this disclosure can be implemented in one or more the components shown in FIG. 3. One or more processing systems can be provided to collectively and/or cooperatively implement the processes and algorithms discussed herein.

As shown in FIG. 3, a processing system in accordance with this disclosure can be implemented using a microprocessor or its equivalent, such as a central processing unit (CPU) and/or at least one application specific processor ASP (not shown). The microprocessor is a circuit that utilizes a computer readable storage medium, such as a memory circuit (e.g., ROM, EPROM, EEPROM, flash memory, static memory, DRAM, SDRAM, and their equivalents), configured to control the microprocessor to perform and/or control the processes and systems of this disclosure. Other storage mediums can be controlled via a controller, such as a disk controller, which can controls a hard disk drive or optical disk drive.

The microprocessor or aspects thereof, in an alternate implementations, can include or exclusively include a logic device for augmenting or fully implementing this disclosure. Such a logic device includes, but is not limited to, an application-specific integrated circuit (ASIC), a field programmable gate array (FPGA), a generic-array of logic (GAL), and their equivalents. The microprocessor can be a separate device or a single processing mechanism. Further, this disclosure can benefit from parallel processing capabilities of a multi-cored CPU.

In another aspect, results of processing in accordance with this disclosure can be displayed via a display controller to a monitor. The display controller preferably includes at least one graphic processing unit, which can be provided by a plurality of graphics processing cores, for improved computational efficiency. Additionally, an I/O (input/output) interface is provided for inputting signals and/or data from microphones, speakers, cameras, a mouse, a keyboard, a touch-based display or pad interface, etc., which can be connected to the I/O interface as a peripheral. For example, a keyboard or a pointing device for controlling parameters of the various processes and algorithms of this disclosure can be connected to the I/O interface to provide additional functionality and configuration options, or control display characteristics. Moreover, the monitor can be provided with a touch-sensitive interface for providing a command/instruction interface.

The above-noted components can be coupled to a network, such as the Internet or a local intranet, via a network interface for the transmission or reception of data, including controllable parameters. A central BUS is provided to connect the above hardware components together and provides at least one path for digital communication there between. It will be understood that terms such as “same,” “equal,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances. The term “substantially” may be used to encompass this meaning, especially when such variations do not materially alter functionality.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

What is claimed is:

1. A temperature modulated probe assembly comprising:
a probe portion for effecting at least one of thermal therapy and cryotherapy to the tissue, the probe portion having a diameter of about 1.65 mm or less, the probe portion comprising: a laser fiber configured for transmitting optical energy along its length, and a diffuser element at a distal tip of the laser fiber and configured for diffusing optical energy received from the distal tip of the laser fiber; and
a depth stop configured to adjust an insertion length of the probe portion within a tissue.

2. The temperature modulated probe assembly of claim 1, wherein the diameter of the probe portion is about 1.1 mm to about 1.65 mm, about 1.15 mm to about 1.6 mm, about 1.2 mm to about 1.55 mm, about 1.25 mm to about 1.5 mm, or about 1.3 mm to about 1.45 mm.

3. The temperature modulated probe assembly of claim 1, wherein the probe portion is configured to emit optical energy with a power output of about 4 watts, about 6 watts, about 8 watts, about 10 watts, or about 12 watts without causing tissue damage outside a target tissue region.

4. The temperature modulated probe assembly of claim 1, wherein the probe portion is configured to ablate lesions having a cross-sectional diameter of about 8 mm to about 26 mm for a single probe trajectory along a probe eye view.

5. The temperature modulated probe assembly of claim 1, wherein the probe portion is configured to ablate lesions having a cross-sectional diameter of about 8 mm to about 10 mm for a single probe trajectory along a probe eye view.

6. The temperature modulated probe assembly of claim 1, further comprising a controller configured to control a temperature of the probe portion using a proportional control algorithm.

7. The temperature modulated probe assembly of claim 6, wherein the controller is configured to control the temperature of the probe portion using the proportional control algorithm to enable optical energy emission for up to about 15 minutes without causing tissue damage outside a target tissue region.

8. The temperature modulated probe assembly of claim 1, wherein the depth stop is a floating depth stop configured to be variably positioned along a length of a probe shaft for adjusting the insertion length of the probe portion within a tissue.

9. The temperature modulated probe assembly of claim 1, wherein the diffuser element is configured to evenly radiate diffused optical energy along an entire length of the diffuser element.

10. The temperature modulated probe assembly of claim 1, wherein the diffuser element is integrally formed at the distal tip of the laser fiber.

11. The temperature modulated probe assembly of claim 1, wherein the probe portion comprises a shaft bonded to a lens.

12. The temperature modulated probe assembly of claim 11, wherein the shaft comprises a shaft lumen for receiving the laser fiber and a cryotherapy generating element that is configured for cooling tissue in proximity of the diffused tip element.

13. The temperature modulated probe assembly of claim 11, wherein the lens forms an enclosed chamber for receiving a distal end of the laser fiber, the diffuser element, and a distal end of the cryotherapy generating element.

14. The temperature modulated probe assembly of claim 13, wherein a distal end of the cryotherapy generating element is proximally offset from a distal end of the laser fiber by a distance of about 0.1-1 mm.

15. The temperature modulated probe assembly of claim 11, wherein the shaft further comprises a temperature sensor configured to measure temperature of a distal section of the probe portion.

16. The temperature modulated probe assembly of claim 15, wherein the temperature sensor proximally offset from a distal end of the laser fiber by a distance of about 1.5 mm to about 3.5 mm.

17. The temperature modulated probe assembly of claim 11, wherein the lens comprises a glass capsule.

18. The temperature modulated probe assembly of claim 11, wherein the shaft is a doubled walled tube comprising an inner tube and an outer tube.

19. The temperature modulated probe assembly of claim 18, wherein the shaft is bonded to the lens by reflowing the outer tube over at least a portion of the lens.

20. The temperature modulated probe assembly of claim 11, further comprising a heat shrink included within the shaft and configured to hold a distal end of the cryotherapy generating element at a stable position with respect to a distal end of the laser fiber.

21. The temperature modulated probe assembly of claim 1, further comprising a cryotherapy generating element comprising a cooling tube having an internal diameter of about 0.250 to 0.350 mm and an outer diameter of about 0.400 to 0.650 mm.

22. The temperature modulated probe assembly of claim 1, further comprising an umbilical sheath for transmitting inputs or outputs between the probe portion and a plurality of probe connectors or the between the probe portion and a control unit.

23. The temperature modulated probe assembly of claim 1, wherein the probe portion comprises gradations for indicating the insertion length of the probe within the tissue.
